(19) United States
(12) Patent Application Publication
(10) Pub. No.: US 2015/0119779 A1
(43) Pub. Date: Apr. 30, 2015

(54) POSTURE LOOP

(71) Applicant: Paul Chen, (US)

(72) Inventor: Paul CHEN, Vancouver (CA)

(73) Assignee: Paul Chen, Vancouver (CA)

(21) Appl. No.: 14/064,269

(22) Filed: Oct. 28, 2013

(51) Int. Cl. A61F 5/02

(52) U.S. Cl. CPC A61F 5/026; A61F 2250/0004
USPC 602/19

(57) ABSTRACT

A posture loop includes an elastic cable having two end portions secured together to form an endless loop, and a retainer bridge engaged between two segments of the elastic cable for separating the segments of the elastic cable from each other and for forming an eight (8)-shaped structure for the posture loop and for engaging onto shoulder portions of a user, a buckle is secured to the first end portion of the elastic cable, and a latch is secured to the second end portion of the elastic cable, and the latch is engageable with the buckle for securing the first end portion and the second end portion of the elastic cable together in order to form an endless loop.

### BACKGROUND OF THE INVENTION

1. Field of the Invention

The present invention relates to an elastic exercise device or back posture training or exercising or straightening device or posture loop, and more particularly to a back posture training or exercising or straightening device or posture loop including an elastic rope or wire or cable supported with a retainer bridge to form an eight (8)-shaped structure or configuration for engaging with or onto the back or shoulder portion of the user and for training or exercising or straightening the shoulder portion and/or the back portion of the user.

2. Description of the Prior Art

Various kinds of typical elastic exercise devices have been developed and provided for conducting or operating various exercise operations and comprise an elastic rope or wire or cable for being operated to exercise various portions or various muscle groups of the user.

For example, U.S. Pat. No. 4,090,706 to Reda, U.S. Pat. No. 4,609,191 to Remme, U.S. Pat. No. 4,733,862 to Miller, U.S. Pat. No. 4,756,527 to Ledbetter, U.S. Pat. No. 4,909,505 to Tee, U.S. Pat. No. 5,318,494 to Santighian, U.S. Pat. No. 5,746,687 to Vial et al., U.S. Pat. No. 5,885,196 to Gvoich, U.S. Pat. No. 6,203,476 to Wang et al., U.S. Pat. No. 6,648,804 to Chen, and U.S. Pat. No. 6,866,617 to Chen disclose several of the typical elastic exercise devices each also comprising an elastic rope or wire or cable for being operated to exercise various portions or various muscle groups of the user.

However, the typical elastic exercise devices may only be pulled or operated or actuated to train or exercise the upper or lower muscle groups, but may not be used to train or exercise or straighten the shoulder portion and/or the back portion of the user.

U.S. Pat. No. 4,852,874 to Sleichter, III et al. disclose another typical elastic exercise device also comprising an elastic rope or wire or cable supported with a retainer ring or sleeve for forming an endless loop or for forming an eight (8)-shaped structure or configuration and for being operated to exercise various portions or various muscle groups of the user, and for training or exercising the upper or lower muscle groups of the user.

However, the typical elastic exercise devices may only be used or pulled or operated or actuated to train or exercise the upper or lower muscle groups of the user, but may not be used to train or exercise or straighten the shoulder portion and/or the back portion of the user.

The present invention has arisen to mitigate and/or obviate the afore-described disadvantages of the conventional elastic exercise devices.

### SUMMARY OF THE INVENTION

The primary objective of the present invention is to provide a posture loop including an elastic rope or wire or cable supported with a retainer bridge for forming an endless loop or for forming an eight (8)-shaped structure or configuration and for training or exercising or straightening the shoulder portion and/or the back portion of the user.

The other objective of the present invention is to provide a posture loop including an elastic rope or wire or cable having two ends adjustably connected together with a buckle for applying an adjustable or changeable resilience or resilient force onto the user, and for allowing the posture loop to be suitably and selectively engaged onto or with the shoulder portions of the users of different sizes or dimensions or standards.

In accordance with one aspect of the invention, there is provided a posture loop comprising an elastic cable including a first end portion and a second end portion, and including an intermediate portion separated into a first segment and a second segment, and a retainer bridge engaged with the elastic cable and engaged between the first and the second segments of the elastic cable for separating the first and the second segments of the elastic cable from each other and for forming an eight (8)-shaped structure for the posture loop and for engaging onto shoulder portions of a user, a buckle is secured to the first end portion of the elastic cable, and a latch is secured to the second end portion of the elastic cable, and the latch is engageable with the buckle for securing the first end portion and the second end portion of the elastic cable together in order to form an endless loop, and for engaging onto and for training or exercising or straightening the shoulder portion and/or the back portion of the user.

The retainer bridge includes an upper pathway and a lower pathway formed therein for engaging with the first segment and the second portion segment of the elastic cable respectively. The buckle includes a cushioning member attached thereto for engaging with the user.

The buckle includes a latch opening formed therein for engaging with the latch. The latch includes a channel formed therein for slidably receiving and engaging with the second end portion of the elastic cable.

A ring may further be provided and engaged with the second end portion of the elastic cable for adjustably attaching the second end portion of the elastic cable to the latch. The ring includes at least one bar provided therein for forming at least one groove therein and for engaging with the second end portion of the elastic cable. The ring includes two bars provided therein for forming three grooves therein and for engaging with the second end portion of the elastic cable.

A pad may further be provided and attached to the intermediate portion of the elastic cable. The pad includes a passage formed therein for slidably receiving and engaging with the intermediate portion of the elastic cable. The pad includes a cushioning member attached thereto for engaging with the user.

Further objectives and advantages of the present invention will become apparent from a careful reading of the detailed description provided hereinbelow, with appropriate reference to the accompanying drawings.

### BRIEF DESCRIPTION OF THE DRAWINGS

FIG. 1 is a rear plan schematic view illustrating the operation of a posture loop in accordance with the present invention;

FIGS. 2, 3 are partial perspective views illustrating the operation of the posture loop;

FIG. 4 is a partial exploded view of the posture loop;

FIG. 5 is a plan schematic view of the posture loop; and

FIG. 6 is a partial cross sectional view of the posture loop, taken along lines 6-6 of FIG. 5; and

FIG. 7 is another partial cross sectional view illustrating the operation of the posture loop.

### DETAILED DESCRIPTION OF THE PREFERRED EMBODIMENT

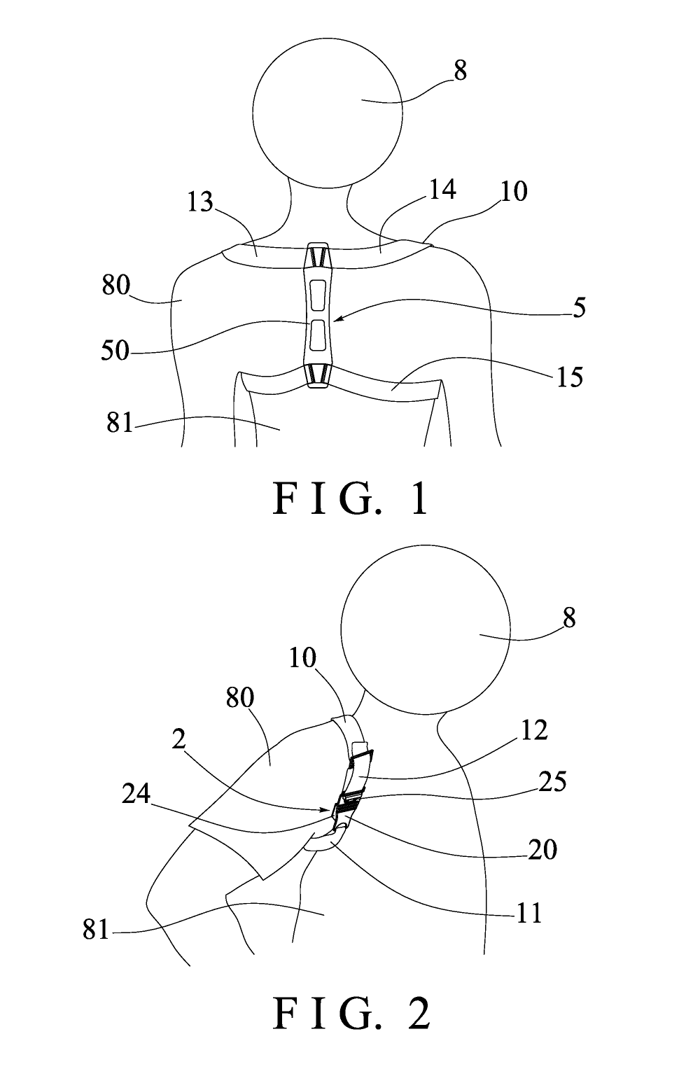

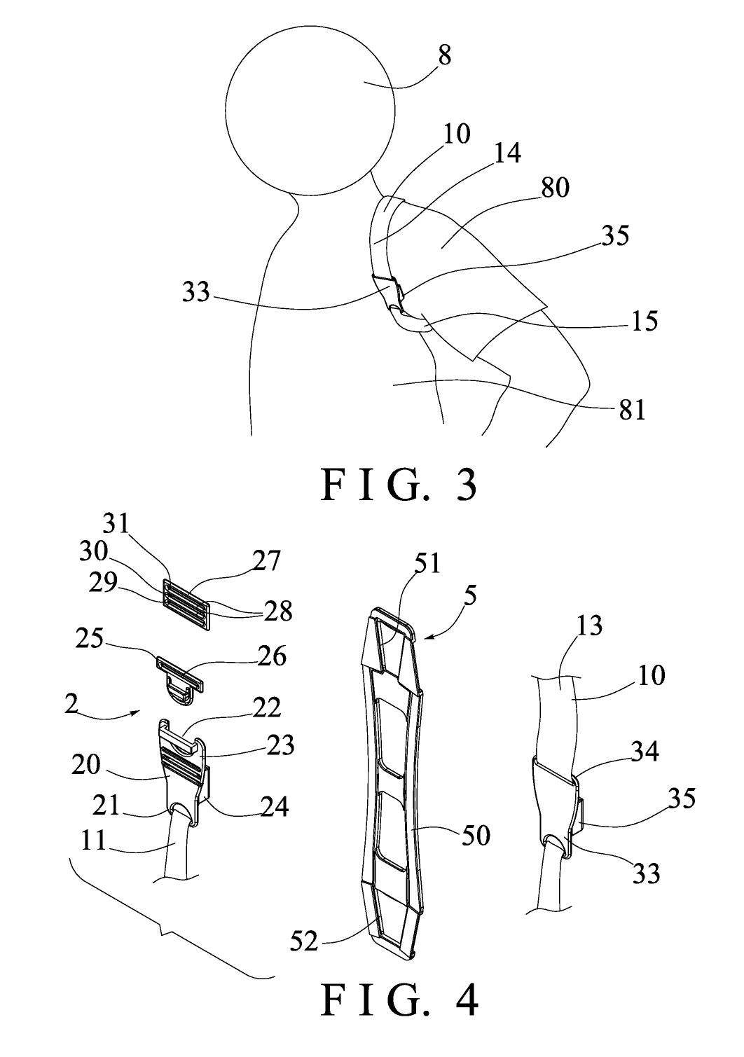

Referring to the drawings, and initially to FIGS. 1-5, a posture loop in accordance with the present invention comprises an elastic rope or wire or band or cable 10 having two ends 11, 12 to be coupled or connected together with a buckle 2 for forming an endless loop and having a middle or intermediate portion 13 coupled or connected together with a retainer bridge 5 for forming an eight (8)-shaped structure or configuration and for engaging with or onto the shoulder portions 80 of a user 8 (FIGS. 1-3), and for training or exercising or straightening the shoulder portion 80 and/or the back portion 81 of the user 8, or for training or correcting the posture of the user 8.

For example, as shown in FIGS. 4-7, the buckle 2 includes a female buckle member or holder 20 having one or first end portion 21 solidly and stably attached or mounted or secured or engaged with or coupled to the one or first end portion 11 of the elastic cable 10 with molding or hot-pressing procedures, adhesives, screws or bolts or catches or latches or fasteners or the like, and having a latch opening 22 formed or provided on the other or second end portion 23 of the holder 20 for detachably or removably receiving or engaging with a male buckle member or latch detent or latch tongue or latch 25 which is provided for engaging with the elastic cable 10 and thus for forming an endless loop or structure or configuration for the elastic cable 10.

The holder 20 includes a pad or cushioning member 24 attached or mounted or secured thereto and made of soft or spongy or resilient materials for softly and comfortably engaging with or onto the shoulder portion 80 of the user 8. The latch 25 includes a passage or pathway or groove or slot or channel 26 formed therein for slidably and adjustably attaching or mounting or securing or latching or locking the other or second end portion 12 of the elastic cable 10 and thus for adjustably attaching or mounting or securing the other or second end portion 12 of the elastic cable 10 to the one or first end portion 11 of the elastic cable 10 with the holder 20 and the latch 25.

Another buckle member or latch element or latch ring 27 may further be provided for adjustably attaching or mounting or securing or coupling the other or second end portion 12 of the elastic cable 10 to the latch 25, and includes one or more (such as two) ribs or beams or bars 28 disposed or provided or engaged therein for forming or defining one or more (such as three) passages or slots or channels or pathways or grooves 29, 30, 31 therein and for threading or engaging with the elastic cable 10. As shown in FIGS. 6 and 7, the other or second end portion 12 of the elastic cable 10 is engageable through one of the grooves 30 and over one of the bars 28 and through the other groove 29 of the ring 27 and then engaged through the channel 26 of the latch 25 and then engaged through the further groove 31 of the ring 27 for adjustably attaching or mounting or securing or coupling the other or second end portion 12 of the elastic cable 10 to the latch 25 and thus for adjusting the length of the endless loop of the elastic cable 10 or the eight (8)-shaped structure or configuration of the posture loop and thus for allowing the posture loop to be suitably and selectively engaged onto or engaged with the shoulder portions 80 and/or the back portion 81 of the users 8 of different sizes or dimensions or standards.

The posture loop further includes another cushioning member or pad 33 having a pathway or channel or groove or slot or passage 34 formed therein for slidably receiving or threading or engaging with the middle or intermediate portion 13 of the elastic cable 10 and for slidably attaching or mounting or securing or retaining the pad 33 to the middle or intermediate portion 13 of the elastic cable 10. The pad 33 includes a cushioning member 35 attached or mounted or secured thereto and also made of soft or spongy or resilient materials for softly and comfortably engaging with or onto the shoulder portion 80 of the user 8. The middle or intermediate portion 13 of the elastic cable 10 may be spaced or separated by the holder 20 and the pad 33 into two portions or segments 14, 15, such as an upper or first portion or segment 14 and a lower or second portion or segment 15.

The retainer bridge 5 is threaded or engaged with or onto the middle or intermediate portion 13 of the elastic cable 10 and disposed or located between the holder 20 and the pad 33 and/or between the segments 14, 15 of the elastic cable 10 for forming an eight (8)-shaped structure or configuration for the posture loop and thus for allowing the posture loop to be attached or mounted or secured or engaged with or onto the shoulder portion 80 and/or the back portion 81 of the user 8. The retainer bridge 5 includes upper and lower grooves or channels or slots or passages or pathways 51, 52 formed therein for slidably receiving or threading or engaging with the middle or intermediate portion 13 of the elastic cable 10, such as the upper or first portion or segment 14 and the lower or second portion or segment 15 of the middle or intermediate portion 13 of the elastic cable 10 respectively.

The retainer bridge 5 may thus be used for extending or spacing or retaining or supporting or disengaging or separating the segments 14, 15 of the elastic cable 10 from each other for thus forming the eight (8)-shaped structure or configuration for the posture loop, and may include a lower base element and an adjustable or movable element (not illustrated) attached or mounted or secured together or engaged with each other for forming or defining an adjustable structure or configuration for the retainer bridge 5 and for allowing the posture loop to be suitably and selectively engaged onto or with the shoulder portions 80 and/or the back portion 81 of the users 8 of different sizes or dimensions or standards.

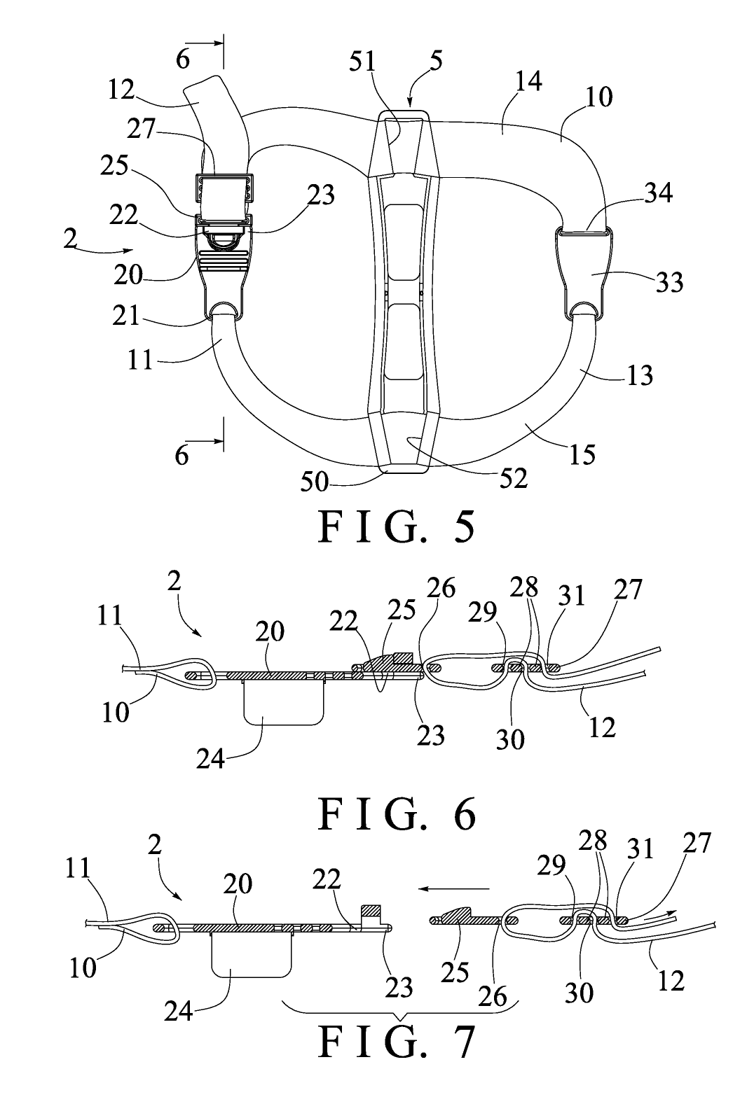

In operation, as shown in FIGS. 1-3, the posture loop includes an eight (8)-shaped structure or configuration for selectively engaging with or onto the shoulder portions 80 of the user 8, and for training or exercising or straightening the shoulder portion 80 and/or the back portion 81 of the user 8. As shown in FIGS. 5-7, the other or second end portion 12 of the elastic cable 10 is movable or adjustable relative to the latch 25 for adjusting the length of the elastic cable 10, and in order to adjust the resilience or spring biasing force of the elastic cable 10 to the user 8, and/or for allowing the posture loop to be suitably and selectively engaged onto or with the shoulder portions 80 and/or the back portion 81 of the users 8 of different sizes or dimensions or standards. The retainer bridge 5 may also include an adjustable structure or configuration for adjusting the distance between the segments 14, 15 of the elastic cable 10 and thus for allowing the posture loop to be suitably and selectively engaged onto or with the shoulder portions 80 and/or the back portion 81 of the users 8 of different sizes or dimensions or standards.

Accordingly, the posture loop in accordance with the present invention includes an elastic rope or wire or cable having two end portions coupled together for forming an endless loop and having a middle or intermediate portion supported with a retainer bridge for forming an eight (8)-shaped structure or configuration and for training or exercising or straightening the back portion of the user, the ends of the elastic rope or wire or cable are adjustably connected together with a buckle for applying an adjustable or changeable resilience or resilient force onto the user.

Although this invention has been described with a certain degree of particularity, it is to be understood that the present disclosure has been made by way of example only and that numerous changes in the detailed construction and the combination and arrangement of parts may be resorted to without departing from the spirit and scope of the invention as hereinafter claimed.

I claim:

1. A posture loop comprising:
an elastic cable including a first end portion and a second end portion, and including an intermediate portion separated into a first segment and a second segment, and
a retainer bridge engaged with said elastic cable and engaged between said first and said second segments of said elastic cable for separating said first and said second segments of said elastic cable from each other and for forming an eight (8)-shaped structure for said posture loop and for engaging onto shoulder portions of a user, wherein
a buckle is secured to said first end portion of said elastic cable, and a latch is secured to said second end portion of said elastic cable, and said latch is engageable with said buckle for securing said first end portion and said second end portion of said elastic cable together in order to form an endless loop.

2. The posture loop as claimed in claim 1, wherein said retainer bridge includes an upper pathway and a lower pathway formed therein for engaging with said first segment and said second portion segment of said elastic cable respectively.

3. The posture loop as claimed in claim 1, wherein said buckle includes a cushioning member attached thereto for engaging with the user.

4. The posture loop as claimed in claim 1, wherein said buckle includes a latch opening formed therein for engaging with said latch.

5. The posture loop as claimed in claim 1, wherein said latch includes a channel formed therein for slidably receiving and engaging with said second end portion of said elastic cable.

6. The posture loop as claimed in claim 1 further comprising a ring engaged with said second end portion of said elastic cable for adjustably attaching said second end portion of said elastic cable to said latch.

7. The posture loop as claimed in claim 6, wherein said ring includes at least one bar provided therein for forming at least one groove therein and for engaging with said second end portion of said elastic cable.

8. The posture loop as claimed in claim 6, wherein said ring includes two bars provided therein for forming three grooves therein and for engaging with said second end portion of said elastic cable.

9. The posture loop as claimed in claim 1 further comprising a pad attached to said intermediate portion of said elastic cable.

10. The posture loop as claimed in claim 9, wherein said pad includes a passage formed therein for slidably receiving and engaging with said intermediate portion of said elastic cable.

11. The posture loop as claimed in claim 14, wherein said pad includes a cushioning member attached thereto for engaging with the user.
